(19) United States
(12) Patent Application Publication
(10) Pub. No.: US 2024/0167006 A1
(43) Pub. Date: May 23, 2024

(54) HIGH PRODUCTION OF RECOMBINANT PROTEIN BY MAKING CELL HYBRIDS AND ENRICHING FOR A PREFERRED MITOCHONDRIAL PHENOTYPE

(71) Applicant: CHO Plus, Inc., South San Francisco, CA (US)

(72) Inventors: Lawrence Form, San Mateo, CA (US); Kathy Ngo, San Francisco, CA (US)

(73) Assignee: CHO Plus, Inc., South San Francisco, CA (US)

(21) Appl. No.: 18/391,547

(22) Filed: Dec. 20, 2023

Related U.S. Application Data
(63) Continuation of application No. PCT/US2023/083811, filed on Dec. 13, 2023.
Application No. PCT/US2023/083811 is a continuation-in-part of application No. PCT/US2023/024973, filed on Jun. 9, 2023.
(60) Provisional application No. 63/350,863, filed on Jun. 9, 2022.

(51) Int. Cl. C12N 9/16; C12N 5/12; C12N 15/02

(52) U.S. Cl. CPC C12N 9/16; C12N 5/12; C12N 15/02; C12N 2510/02; C12Y 301/03001

(57) ABSTRACT

This disclosure provides a technology for producing cells and cell lines that are capable of manufacturing a recombinant protein more efficiently and in a more cost effective manner. Cells are engineered or fused together to make a population of cell hybrids. The hybrids are then enriched for a mitochondria phenotype that supports and enhances protein synthesis and secretion. The phenotype may include a higher content of mitochondria per cell, a high or low cellular content of reactive oxygen species (ROS), and/or a high or low mitochondria membrane potential. The hybrids may also be enriched for a high content of endoplasmic reticulum. Producer cell lines obtained in this fashion may produce as much as 10 or 15 times the amount of protein per cell than the parental cell line. The producer cells can be used for efficient manufacture of recombinant proteins suitable for use in any industrial process, such as the making of pharmaceutical agents and food ingredients.

### RELATED APPLICATIONS

This application is a continuation of international patent application PCT/US2023/083811, filed Dec. 13, 2023 (pending). This application is also a continuation-in-part of international patent application PCT/US2023/024973, filed Jun. 9, 2023 (pending), published as WO/2023/239928 on Dec. 13, 2023, which claims the priority benefit of U.S. provisional patent application 63/350,863, filed Jun. 9, 2022. The aforesaid priority applications are hereby incorporated herein by reference in their entireties for all purposes.

### TECHNICAL FIELD

This disclosure relates generally to the production of pharmaceutical compounds and other industrial products that contain recombinant protein. It also relates to the modification, selection, and genetic alteration of host cells for high levels of production of pharmaceutical products with improved biological and pharmacological characteristics.

### BACKGROUND

Biological agents constitute a continually growing proportion of the market for pharmaceuticals. They usually have better specificity than small molecule drugs, leading to more targeted efficacy with fewer side effects. However, use of biological agents emphasizes a need for improved means of industrial production, with greater productivity and lower costs.

Monoclonal antibodies have proved to be powerful therapeutic agents for the treatment of a myriad of diseases including cancer, autoimmune, and infectious diseases. Biological drugs designed to promote hemostasis, particularly blood clotting factors, are projected to achieve a market size of $47 billion by 2030.

There are now more than about 200 biological agents approved for use in therapy by the U.S. Food and Drug Administration. Current levels of industrial recombinant antibody production are generally no more than 4 g per liter of culture fluid: more typically less than 2 g per liter. Some therapeutic proteins have a dose and administration schedule that uses more than 10 grams of protein per patient per year. To supply the market for a particular protein product, it may be necessary to produce as much as 400,000 kg of that protein per year.

The owners of the technology described below previously developed a system for increasing production of monoclonal antibodies in producer cell lines by over four-fold. U.S. Pat. No. 10,329,594. Cultured cells such as CHO cells are fused together, and hybrids are selected for a high content of endoplasmic reticulum or Golgi apparatus. The technology described in this disclosure provide a further advance in the technology that further increases the amount of protein that can be produced in cell culture.

### SUMMARY OF THE INVENTION

This disclosure provides a technology for producing cells and cell lines that are capable of manufacturing a recombinant protein more efficiently and in a more cost effective manner. Cells are engineered or fused together to make a population of cell hybrids. The hybrids are then enriched for a mitochondria phenotype that supports and enhances protein synthesis and secretion.

The phenotype may include a higher content of mitochondria per cell, a high or low cellular content of reactive oxygen species (ROS), and/or a high or low mitochondria membrane potential. The hybrids may also be enriched for a high content of endoplasmic reticulum (ER). Producer cell lines obtained in this fashion may produce as much as 10 or 15 times the amount of protein per cell than the parental cell line. The producer cells can be used for efficient manufacture of recombinant proteins suitable for use in any industrial process, such as the making of pharmaceutical agents and food ingredients.

An objective of this disclosure is to provide technology for producing recombinant proteins at a high production capacity per cell. As an overview, the method typically comprises providing a starter population of cultured cells, forming cell hybrids therefrom, each comprising two or more cells, enriching the cells for a particular mitochondria phenotype, genetically altering the cells to express the target protein, and the culturing the genetically altered cells to produce the target protein.

The optimal mitochondria phenotype may comprise a high or low content of mitochondria per cell, a high or low reactive oxygen species (ROS) per cell, or a high or low mitochondria membrane potential per cell, or any two of these in combination, or all three. As demonstrated in the examples below, the optimal mitochondria phenotype may differ between different cell lines, different culture conditions, and different recombinant proteins being produced. As an initial test of a given system, the user may wish to try several permutations: for example, high mitochondria level per cell, high and alternatively low ROS, each with high and alternatively low mitochondria membrane potential. After testing protein production from a transgene in the various cell population obtained, the cells with the best phenotype may be used to obtain a producer cell line—or the entire cell deriving process may be repeated, knowing which phenotype choices are optimal.

As an optional step, the technology may also include enriching the cells for a high content of endoplasmic reticulum (ER) and/or Golgi apparatus per cell, either before or after optimizing the mitochondria phenotype.

An aspect of this disclosure is a process for establishing a producer cell line for high efficiency production of a target protein. The process typically comprises forming a population of cell hybrids from a starter cell population, each comprising two or more cells; then testing and selecting cell hybrids for mitochondria phenotype, including mitochondria content per cell, reactive oxygen species (ROS) per cell, and/or mitochondria membrane potential per cell. The selected and recovered cells are cultured and expanded to establish the producer cell line.

As before, the optimal mitochondria phenotype may include a higher or lower content of mitochondria per cell, a higher or lower content of reactive oxygen species (ROS) per cell, and/or a higher or lower mitochondria membrane potential per cell in any combination. Selection is conveniently but not necessarily done by staining cells with a dye that indicates the desired feature, and then sorting for the appropriate dye (either high or low level) using a cell sorter. Optionally the cell hybrids may also be selected that have a higher density of endoplasmic reticulum (ER) or Golgi apparatus per cell.

At some point in the process, the cells are usually selected for protein production capacity (usually from a transgene inside the cell encoding the target protein or a marker, but possibly an endogenous protein). The transgene may encode, for example, the target protein, or a proxy therefor in the form of a marker protein. The cells may also be selected for rapid growth, for example, by an iterative dilution technique that permits only fast growing cells to survive.

The cells can be aliquoted or cloned (for example, by limiting dilution) at any point in the process following creation of the hybrids: before or after sorting for mitochondria phenotype, before or after sorting for ER content, and before or after selecting for high protein prediction capacity per cell. They may be recloned at several stages of the process to stabilize a desired phenotype.

The cells may be genetically altered to express a transgene that encodes a recombinant protein, such as a marker protein, or the target protein at any point in the process: as early as the starter cell population, or after sorting for mitochondria phenotype, or after sorting for ER, or after sorting for high protein production, or after sorting for rapid growth rate. The producer cells may also be banked without a transgene, which can be introduced later.

The starter population used to create fused cells or cell hybrids is typically a single cell line, such as CHO cells, mouse myeloma NSO cells, mouse myeloma SP2/0 cells, human embryonic kidney 293 (HEK 293) cells, baby hamster kidney 21 (BHK-21) cells, VERO cells, PER.C6 cells, or HeLa cells. Cell mixtures may also be suitable, including autotypic hybrids, mixtures of more than one cell line, or primary cells optionally in combination with an established cell line.

This disclosure provides a means of improving cells or a cell derivation protocol to further enhance protein production capacity. For example, for cell populations already fused and/or enriched for cell concentration of ER, sorting for mitochondria phenotype may be an add-on step that boosts productivity further. Alternatively, the sorting for mitochondria phenotype may occur shortly after cells are fused, without sorting for ER.

As a result of implementing this technology, the user may obtain an established line of producer cells wherein each cell is a hybrid of more parental cell lines, wherein the producer cells comprise an expressible transgene that encodes a target protein, and contain more mitochondria and/or reactive oxygen species (ROS) per cell than any of the parental cell lines.

In any of the contexts put forth above, the target protein may be a pharmaceutical agent such as an antibody or portion thereof, a food ingredient, or an enzyme or other protein useful in mass quantities in another industry. A producer cell containing a transgene encoding the target protein may be cultured under conditions whereby the protein is expressed, from which it may be harvested and purified. If the target protein is a pharmaceutical agent such as an antibody, it may be formulated and compounded to produce the pharmaceutical product in a manner such that the product is suitable for human administration.

Throughout this disclosure, the terms “fused cells”, “cell hybrids”, and “engineered cells” refer interchangeably to a cell made by combining two or more parental cells together to create a single cell bearing organelles and at least some chromosomes from all parents within a combined plasma membrane. Since the shared cells are quadriploid, they shed some of their chromosomes, but retain others and are thereby distinguishable from the starting cell population.

The term “target protein” refers to a recombinant protein or combination thereof that is targeted for industrial scale production according to this disclosure. A transgene encoding the target protein can be transfected into the hybrid cells during the course of selection of the desired cell phonotype. Alternatively, the transgene can be transfected into a banked line of producer cells already established as being capable of producing recombinant protein at a high level. In reference to cell populations, the verb “selecting” encompasses all sensible manners of separating one cell phenotype from another. Depending on context, it may include separating using a cell sorter, separating using an antibody column, or selecting by outgrowing a desired subset of cells in culture. Cells that have been “genetically altered” or “transfected” have incorporated a transgene by viral or non-viral means so that the encoded protein may be expressed transiently or after integration into the cell genome.

Various aspects, embodiments, features, and characteristics of the invention are described in the sections that follow, the accompanying drawings, and the appended claims.

### BRIEF DESCRIPTION OF THE DRAWINGS

FIG. 1 is a workflow that can be used for obtaining hybrid cell lines with high protein production capacity. Cells are fused together and optionally selected for high endoplasmic reticulum (ER) content in one or more cycles. The hybrids are selected for one or more mitochondria phenotypes, thereby obtaining hybrids with a preferred phenotype. The cells can be cloned to stabilize the phenotype, and selected for high protein expression from a transgene encoding a marker or the target protein. Clones obtained in this fashion are expanded and banked for production of pharmaceutical agents and other industrial recombinant proteins.

FIGS. 2A and 2B show production of recombinant protein by fused and sorted producer cells established from CHO cells and HEK 293 cell lines, respectively. FIG. 2A shows the cumulative volumetric productivity of trastuzumab (Herceptin®) from transgenes that were transiently transfected into either of two cell populations: (1) the starting CHO-K1 cells; and (2) a clone of hybridized CHO-derived cells that were selected for high endoplasmic reticulum, high mitochondria and low reactive oxygen species (ROS) (n=3). Selecting for these criteria yielded cells with production capacity that was a 15-fold improvement compared with the starting cells.

FIG. 2B shows the cumulative volumetric productivity of trastuzumab (Herceptin®) from transgenes that were transiently transfected into either of two cell populations: (1) a starting HEK 293 cells; and (2) a clone of hybridized HEK 293 cells that were selected for high mitochondria content and high reactive oxygen species (ROS) instead of low ROS (n=3). This yielded a 10-fold improvement in protein production by the selected cells, compared with the un-engineered parent cell line. In this instance, there was no screening for high ER. This shows that screening for mitochondria phenotype alone is sufficient to obtain producer cells with substantially increased protein production capacity.

FIG. 3 is a workflow used to screen fused HEK 293 cells according to intracellular content of mitochondria and reactive oxygen species (ROS).

FIG. 4 shows protein production from a population of CHO cells fused and selected for high content of endoplasmic reticulum (but not mitochondria phenotype). The protein production is 4.5-fold better compared with parental CHO cells. Both populations were transfected to express secreted alkaline phosphatase (SEAP).

FIGS. 5A, 5B, and 5C demonstrate higher productivity of viral capsids of several serotypes of AAV vectors from fused HEK 293 cells. A selected clone showed an increase in AAV vectors produced compared with parent host.

FIGS. 6A and 6B. demonstrate higher functional titer of several serotypes and other features of AAV vectors from fused HEK 293 cells.

FIG. 7 demonstrate an improvement in the proportion of capsids containing a nucleic acid payload achieved by screening fused cells for cell phenotype.

### DETAILED DESCRIPTION

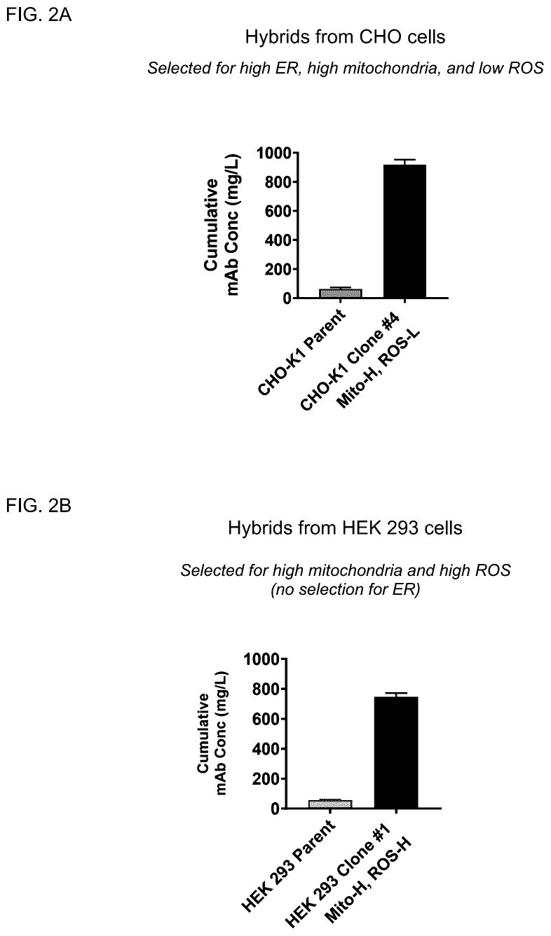

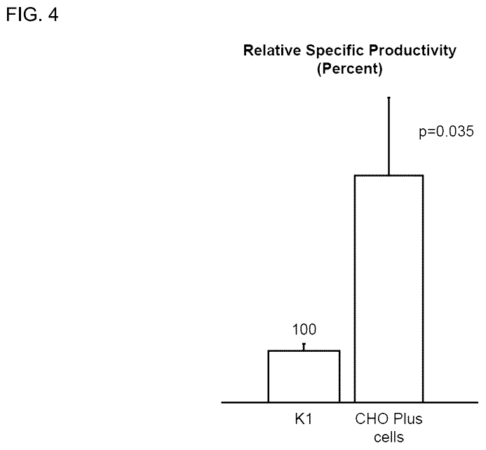

This disclosure provides improved cell lines for manufacture of recombinant protein. Fusing cells and sorting for high intracellular content of endoplasmic reticulum (FIG. 4) creates producer cells that produce about 4-fold more protein per cell than the parental cells. Selecting mitochondria phenotype as well (FIGS. 2A and 2B) can yield cells that produce as much as 10-fold or 15-fold more protein per cell than the parental cells.

### 1. Overview

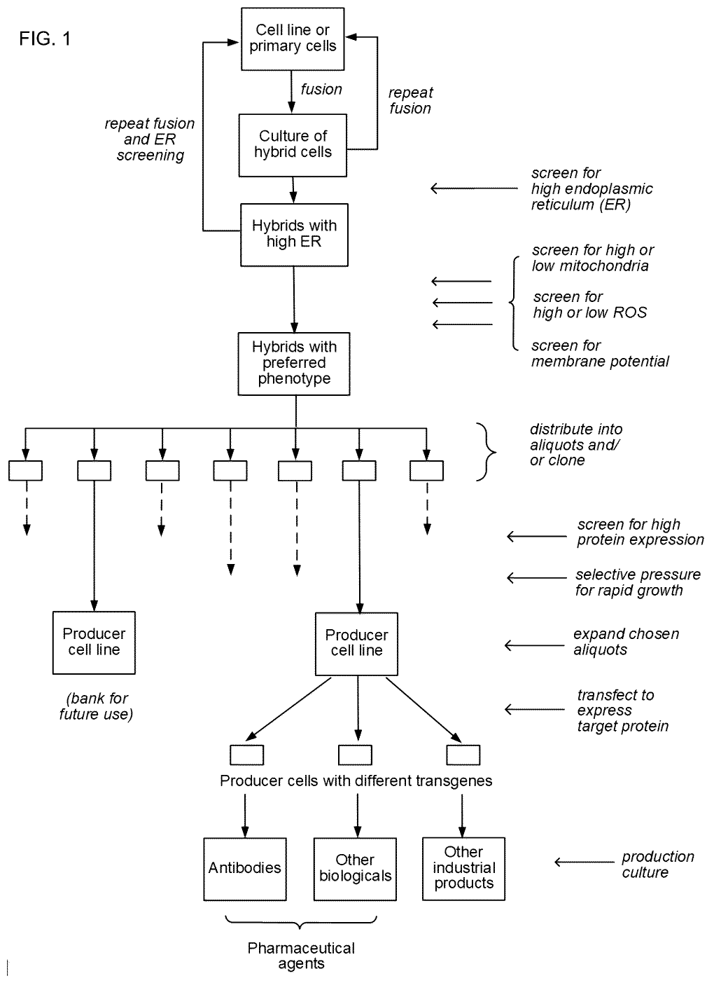

FIG. 1 illustrates a suitable workflow by which the technology of this disclosure can be implemented. The starting cell population shown at the top is treated so that at least some cells in the population produce cell hybrids comprising at least two of the starting cells. The hybrid cells are optionally sorted for a high intracellular content of endoplasmic reticulum (ER) using an ER specific stain. Hybrids containing the highest ER content are harvested, for example, using a cell sorter. It is often useful to do multiple fusion cycles, with or without an ER sorting step in each cycle.

This diagram shows the hybrids subsequently being sorted for mitochondria phenotype: specifically, any one, any two, or all three of the following: (1) the content of mitochondria per cell, (2) the content of reactive oxygen species (ROS), and (3) mitochondria membrane potential. This is done conveniently by using a dye specific for each feature, then sorting and recovering the highest staining cells using a cell sorter. Sorting for either high or low ROS and high or low membrane potential may enrich for high producer cells, depending (amongst other things) on the original cell line and the target protein to be manufactured. The user may wish to try both the top end and the bottom end of each feature to determine which works best for the particular cell line being used, and the ultimate objective.

After sorting for mitochondria phenotype, the diagram shows the selected cells being aliquoted or cloned to preserve the preferred cell phenotype. Cloning can be done before or after sorting for mitochondria phenotype, or both. High producer clones can be identified at any stage in the process by detecting protein expression. This can be done by genetically altering the entire cloned or aliquoted population to express a transgene, or by testing just a sample of each clone or aliquot. Once the high producer clones are identified, the transfected samples may be discarded so that the best clones or aliquots can be banked in untransfected form.

Cells can also be selected for rapid growth. This can conveniently be done, for example, by diluting cells in the culture medium (at the time of or in between cell passages). Cells that grow faster out-compete the slower cells, and come to predominate the cell population.

The resulting cell population can be expanded and banked—either genetically altered to express the target protein, or without a transgene as a resource for expressing other target proteins at a future time. Producer cell lines obtained according to this disclosure can be used, for example, to produce pharmaceutical agents such as specific antibody, or for other industrial uses such as food ingredients.

What is shown in FIG. 1 is analogous to a tool kit. The user may employ the steps and technology shown in any workable order, with each step being optional, unless explicitly stated or otherwise required.

### 2. Rationale

High producer cells of a target protein are obtained by making cell hybrids, and then selecting hybrids that have a desirable phenotype for efficient protein production. As described in the sections that follow, mitochondrial phenotype is optimized because mitochondria play several critical roles in the production of protein by a cell.

The endoplasmic reticulum (ER) and mitochondria both participate in the biosynthesis and secretion of protein by a cell. Mitochondria associated membranes (MAM) create transient contact sites between the ER and mitochondria. The contact sites provide calcium microdomains for cellular signaling such as activation of Ca⁺⁺-dependent metabolic enzymes. The contact sites are rich in specific proteins and lipids that facilitate communication and functional crosstalk, largely facilitated by Mitofusin 1 and 2. Evidence of ER-to-mitochondria trafficking has been reported for a number of proteins. This includes activation of the unfolded protein response (UPR) that can enhance protein secretion. C. Koch et al., Int J Mol Sci. 2021 September; 22(17): 9655, V. Derderer, Elife. 2019 Jun. 7:8:e45506.

Reactive oxygen species (ROS) originating from mitochondria cause changes in the in vivo redox status of cells including several factors involved in the regulation of protein synthesis. (Topf et al., Nat Commun. 2018; 9: 324, 2018 Jan. 22). Redox switches have been implicated in a global protein synthesis, wherein mitochondria-derived oxidative stress can attenuate protein synthesis.

By enriching for a particular mitochondria phenotype, hybrid cells can be selected that have improved efficiency for production of a target protein.

### 3. Benefits of this Technology

Depending on the mode of practice and application, aspects of this disclosure described in this disclosure can be used to select cell hybrids that produce a higher concentration of a target protein per volume of culture fluid. This in turn has the following benefits.
reduces the cost of production of recombinant protein for clinical use, thereby improving access to such therapeutic agents;
reduces the need to enlarge or build new GMP production facilities as market size increases;
provides for GMP production of kilogram quantities of finished protein product with relatively small or fewer bioreactors;
creates established producer cell lines suitable for high-level expression of new target proteins, as needed;
decreases cloning or selection steps that are needed following integration of the gene to be expressed;
improves product quality; and
provides high quality low volume research materials, thereby reducing the time needed to initiate and complete clinical trials.

### 4. Demonstration of the Effectiveness of Enriching Cells for Mitochondrial Phenotype

Enriching for an optimal mitochondrial phenotype can increase the amount of recombinant protein produced per cell by as much as 10- to 15-fold. This is illustrated by the data shown in FIGS. 2A and 2B.

FIG. 2A shows the results of a protocol in which CHO-K1 cells were repeatedly hybridized and selected for high levels of endoplasmic reticulum, then selected for high numbers of mitochondria per cell and low levels of reactive oxygen species (ROS).

Briefly, CHO-K1 cells were fused or hybridized using polyethylene glycol (PEG) 1500 as fusogenic agent. The cell hybrids were first screened for a high intracellular content of endoplasmic reticulum (ER) using ER Tracker Green dye. The top 10% of stained cells were obtained by cell sorting, and then expanded Five cycles of cell fusion, ER sorting, and expansion were done consecutively, selecting the top 10% expressors each time. After the fifth fusion, the cells were cloned. The clones were sampled for high protein production by transient transfection to express both chains of trastuzumab (marketed as Herceptin®).

The highest producer cloned was then expanded and sorted for high mitochondria content using Biotracker 405 Blue Mitochondria. The cells were also sorted for low reactive oxygen species (ROS) and low mitochondria membrane potential using CellROX Green, and tetramethylrhodamine ethyl ester perchlorate (TMRE) simultaneously. The cells were recloned, resampled, and retested for expression of trastuzumab.

The data show the cumulative volumetric productivity (mg/L) of trastuzumab following transfection into (1) the parent CHO cells; or (2) the clone of hybrid cells that had been selected for high ER and a particular mitochondria phonotype: high content of mitochondria, and low reactive oxygen species (ROS) (n=3). Selecting for this phenotype yielded a 15-fold improvement in protein production, compared with the un-engineered parent cell line.

FIG. 2B was obtained from HEK 293 cells that went through seven rounds of fusion, followed by selection for high mitochondria level and high instead of low ROS. The graph shows cumulative productivity (mg/L) of trastuzumab from (1) the starting population of HEK 293 cells; or (2) the final clone of fused and selected HEK 293 cells (n=3). In this instance, selecting for high mitochondria and high mitochondria yielded a 10-fold improvement in protein reduction. In this instance, there was no screening for high ER. This shows that screening for mitochondria phenotype alone is sufficient to obtain producer cells with increased protein production capacity.

Details of the protocol used to obtain the data shown in FIGS. 2A and 2B are provided in Example 4 (below).

### 5. Different Approaches for Making Cell Hybrids

Individual high producer cells can be selected from any cell population, such as one or a combination of cell lines and/or primary cells. Many single cell lines (such as CHO and HEK 293 cells) are sufficiently diverse at the outset in terms of gene content and intracellular apparatus in the proliferating cell population that they can be sorted and selected for high producer cells directly from a standard culture.

Optionally, to improve final product yield or enhance the sorting process, the user may prepare cells for sorting by taking one or a combination of techniques that will either enhance heterogeneity of levels of protein production within the cell population, or generally increase the levels of protein production for the cells population as a whole, or a subpopulation thereof. Suitable techniques are those that alter the genome of the cells, for example, to shuffle the genome and increase copy numbers of that contribute to the intracellular machinery involved in protein production and secretion. Altering or shuffling the genome in this manner may yield many genetic variants with one or more of a variety of different properties, including levels of target protein production and growth rate.

The technology of this disclosure is based on part on the disclosure that cells suitable for protein production can attain a higher level of production by fusing with other cells. Without limiting practice of the invention, it is hypothesized that fusing two cells together is partly additive in terms of the components, genetics, or genetic control of the cells that participate in protein production. It is beneficial if the improved characteristics breed true. Accordingly, after cells are fused, they are typically subject to multiple rounds of culturing and selection for phenotypic characteristics of interest. The resulting cells may be aneuploid or otherwise retain all or part of the genomes of parental cells that encode cell components involved in protein production.

Model cells suitable for fusion are cell lines that have already been employed for industrial protein production, such as CHO cells, mouse myeloma NSO cells, mouse myeloma SP2/0 cells, rat myeloma YB2/0 cells, Human Embryonic Kidney (HEK) 293 cells, HeLa, Per.C6, HT-1080, Huh-7, Baby Hamster Kidney (BHK-21), and Per-CP cells. In the context of this disclosure, a “cell line” is a population of cells that can be propagated continually, extensively, or indefinitely in tissue culture. A starting cell line is typically heterogeneous in terms of one or more phenotypic features that relate to the amount of gene product from a transgene that the cell will produce. When cultured, a producer cell line obtained according to this disclosure may produce progeny that are heterogeneous, substantially homogeneous, or clonal.

Cell fusion is performed by obtaining a cell mixture of cells to be fused: (a plurality of cells from one cell line, or more than one cell line, or a mixture of at least one cell line and at least one primary cell population). The cell mixture is then subjected to an appropriate fusion protocol: for example, by culturing under culture conditions that promote the formation of hybrids, by conducting an electrofusion, by combining with a fusogenic virus such as Sendai virus, by placing cells into contact (for example, by gentle centrifugation), by treating with a fusogenic agent such as polyethylene glycol (PEG), or using any effective combination thereof.

For purposes of this disclosure, cells that have been made by fusing two or more cells together may be referred to as autotypic hybrids (cells from the same cell line fused together), isotypic hybrids (cells having the same genotype), allotypic hybrids (cells from different individuals of the same species having different genotypes), and xenotypic hybrids (cells from different species). Autotypic hybrids are typically formed using a population of cells that consists essentially (that is, at least 99%) of cells from a single cell line. The other types of hybrids are typically formed using cell populations from two or more cell lines which have potentially complementary properties. The disclosure also includes the fusion of one or more cell populations isolated or obtained from primary sources with themselves or with established or cloned cell lines.

Cells may be fused into hybrids using any suitable technique. For example, cells may be cultured in the presence of a fusogenic agent and/or under culture conditions that promote the formation of hybrids, or may be forced into contact, for example, by gentle centrifugation, optionally in combination with a fusogenic agent such as polyethylene glycol (PEG). Typically, a fused cell is obtained by fusing two cells together, although fusion of three or more cells is possible. It is recognized that fusion of two different cell populations will result in mixed cell products (isotopic, allotypic, or xenotypic hybrids, depending on the parental cell lines), and autotypic hybrids. Autotypic or isotopic hybrids can be separated from allotypic or xenotypic hybrids, if desired, using fluorescently labeled or surface bound antibody specific for a ligand expressed on one of the cell lines in the mixture, but not another.

All such combinations come within the scope of this invention, unless explicitly indicated otherwise. It may be beneficial to repeat the cell fusion within a population of hybrids to enhance the effect further, and/or cross-hybridize with other cell lines to imbue the ultimate cell line with additional beneficial characteristics. Thus, the fusion and selection steps may be done iteratively twice, three or four times, or more.

### 6. Methods and Reagents for Enriching Cellular Content of Endoplasmic Reticulum (ER) and/or Golgi Apparatus

Subcellular organelles involved in production of protein include the endoplasmic reticulum (ER) and the Golgi apparatus. Either or both of these can be measured and used as a basis for sorting or selection without damaging the cell using a vital dye, and the cells can be selected on the basis of the amount of dye that is associated.

Such dyes can be obtained commercially, for example from the company Molecular Probes. Examples of vital dyes for ER include:
ER-Tracker™ Blue-White DPX (E12353)
ER Tracker™ Green (glibenclamide BODIPY® FL) (E34251)
ER-Tracker™ Red (glibenclamide BODIPY® TR) (34250)
DiOC6 (D273)
Di005 (D272).
   Vital dies for Golgi apparatus include:
NBD C6-6-ceramide (N1154)
NBD C6-sphingomyelin
BODIPY® FL C5-cerimide (D3521)
BODIPY® TR ceramide (D7540)

Alternatively or in addition, the user can test expression-based labeling systems that would introduce a fluorescent protein targeted to ER or Golgi. They are fusion proteins comprising a portion that expresses an optical label, fused with a protein sequence that targets or is processed by the organelle to be labeled. Examples include the following:

Invitrogen:
CellLight™ ER-GFP (C10590)
CellLight™ ER-GFP (C10591)
CellLight™ Golgi-GFP (C10592)
CellLight™ Golgi-GFP (C10593)

### Evrogen:

pmKate2-ER (FP324)
pFusionRed-ER (FP420)
pTagRFP-Golgi (FP367)
pFusionRed-Golgi (FP419)

### Clontech:

pDsRed2-ER Vector (632409)
pDsRed-Monomer-Golgi Vector (632480)
pAcGFP1-Golgi Vector (632464)

After staining with any of these dyes, cells may be selected (for example, by flow cytometry and sorting) that have on average a level of staining that is at least 1.2, 1.5, 2, or more than 2-fold higher than the parental cell line or lines, in terms of staining, for example, for ER, Golgi, or an optically labeled gene product.

### 7. Methods for Enriching Cellular Content of Mitochondria, Reactive Oxygen Species (ROS), and Membrane Potential

Mitochondria content and function can be used as basis for sorting, selection, and/or characterization without damaging the cell by using vital dyes. Such dyes can be obtained commercially, for example from the companies Invitrogen and Sigma Aldrich. Examples of vital dyes for the mitochondria include MitoTracker Green FM; MitoTracker Orange CMTMRos; MitoTracker Red CMXRos; MitoTracker Red FM; MitoTracker Deep Red FM; BioTracker 488 Green Mitochondria dye; BioTracker 633 Red Mitochondria dye; BioTracker 405; and Blue Mitochondria.

Functional dyes to measure the membrane or redox potential of the mitochondria can also be used to sort or select for cells with enhanced mitochondria function. Mitochondria potential is generated by Complexes I, III and IV and serves as a reliable read-out to assess mitochondria function. Membrane depolarization shifts fluorescence signal from one wavelength to another. These membrane potential dyes are available from companies: Invitrogen and Sigma Aldrich: JC-1 Dye (Invitrogen T3168; Sigma CS0390); JC-9 Dye (Invitrogen D-22421); and C10 Dye (Sigma MAK160, MAK159).

Additional characteristics to sort for enhanced mitochondria includes vital dyes to measure mitochondria calcium, superoxide production, and dyes selective to the mitochondria. These include: Rhod-2 AM Reagent (Invitrogen R1245MP); and MitoSOX Red (Invitrogen M36008).

Alternatively or in addition, the user can test expression-based labeling systems that would introduce a fluorescent protein targeted to the mitochondria. They are fusion proteins comprising a portion that expresses an optical label, fused with a protein sequence that targets or is processed by the organelle to be labeled. Examples include the following. From Invitrogen: CellLight™ Mitochondria-GFP (C10600); and CellLight™ Mitochondria-RFP (C10505, C10601). From Evrogen: pTagCFP-mito (FP117); pTagYFP-mito (FP137); pTagRFP-mito (FP147); pmKate-mito (FP187); pTagGFP2-mito (FP197); pTurboRFP-mito (FP237); pTurboGFP-mito (FP517); pPhi-Yellow-mito (FP607); and pTurboFP602-mito (FP717). From Takara Bio: pAcGFP1-Mito Vector (632432); pDsRed2-Mito Vector (632421); pHcRedl-Mito Vector (632434); and pPAmCherry-Mito Vector (632591);

After staining with any of these dyes, cells may be selected (for example, by flow cytometry and sorting) that have on average a level of staining that is at least 1.2, 1.5, 2, or more than 2-fold higher than the parental cell line or lines, in terms of staining, for example, for mitochondria or an optically labeled gene product.

The intensity of staining reflects a relative “amount”, “concentration”, “level” or “content” of mitochondria per cell. A high content of mitochondria may reflect a higher level of mitochondria organelles, or larger sized mitochondria, or a combination of both.

Cellular reactive oxygen species. This disclosure demonstrates for the first time that cellular content of reactive oxygen species can provide a selection basis for increased protein production capability. The effect is dependent inter alia on the starter cell line used to prepare hybrids. A higher cellular content of ROS correlates with enhanced production of target protein in HEK 293 cells. However, a lower content of ROS correlates with enhanced production in CHO cells. Reactive oxygen species (ROS), such as superoxide anion (O²⁻), hydrogen peroxide (H₂O₂), and hydroxy radical (HO·), constitute radical and non-radical oxygen species formed by partial reduction of oxygen. Cellular ROS are generated endogenously as in the process of mitochondria oxidative phosphorylation and have been implicated in a variety of pathological diseases such as cancer, neurodegeneration, and aging.

Cellular reactive oxygen species can be measured using fluorescent probes, wherein upon oxidation, these reagents exhibit strong fluorescence and remain localized within the cell. These dyes are commercially available and include the following: From ThennoFisher: CellROX® Green; CellROX® Orange; CellROX® Deep Red; and H2DCFDA. From Abcam: DHE (Dihydroethidium) Assay Kit.

Intracellular redox levels can be determined using OxyBURST Green reagents, RedoxSensor Red CC-1 stain, and reduced calcein, ethidium, fluoresceins, MitoTraker probes, and rhodamines. Intracellular pH can be determined using 9-amino-6-chloro-2-methoxyacridine (ACME), BCECF indicator, dextran conjugates, fluorescein and fluorescein derivatives, 8-hydroxypyrene-1,3,6-trisulfonic acid (HPTS), LysoSensor probes, Oregon Green dyes, pHrodo indicator, SNARF indicator, and thiol-reactive pH indicators. Other protein reagent for the detection and quantitation of ROS are CellROX Deep Red, CellROX Green and CellROX Orange from Invitrogen Molecular Probes. Each reagent is cell-permeant and is nonfluorescent or very weakly fluorescent in the reduced state. Upon oxidation, the reagents exhibit strong fluorescence and remain localized within the cell.

Mitochondria Membrane Potential (MMP or ΔΨm) is a feature that maintains the electrochemical potential of hydrogen ions needed to synthesize ATP via the electron transport chain and has been shown to be an important factor in mitochondrial health. The MMP influences the balance between ATP production and other cellular processes that consume ATP. Under normal physiological activity, MMP remains relatively stable. However, long-term disturbance has detrimental effects on cellular health and viability and leads to various pathophysiologies. Monitoring MMP provides a sensitive and early indicator of mitochondrial integrity and is crucial in identifying mitochondrial dysfunction related to pathological diseases such as diabetes, cancer, and neurodegeneration. Live cell imaging of mitochondria following targeted irradiation in situ reveals rapid and highly localized loss of membrane potential. DWM Walsh et al., Scientific Reports volume 7:46684 (2017).

The Incucyte® Mitochondrial Membrane Potential Assay (Sartorius) can be used for real-time detection of transient and long-term changes in MMP in live cells. Alternatively, the TMRE-Mitochondrial Membrane Potential Assay Kit (Abeam), the MitoPT TMRE Assay (Immunochemistry Technologies), or the Mitochondrial Membrane Potential MitoPT™ TMRE Kit (BioRad) can be used for quantifying changes in mitochondrial membrane potential in live cells by flow cytometry, microplate spectrophotometry and fluorescent microscopy. TMRE (tetramethylrhodamine ethyl ester) is a cell permeant, positively-charged, red-orange dye that readily accumulates in active mitochondria due to their relative negative charge. Depolarized or inactive mitochondria have decreased membrane potential and fail to sequester TMRE.

### 8. Characterizing High Producer Cell Lines

Cell hybrid cells that have been optimized for the production of a target protein can be characterized by one or more criteria in any combination.

Suitable criteria include cell karyotype. Chromosome patterns can be characteristic of homotypic and heterotypic cell fusions. The following characteristics may be favorable for protein production:
duplication of chromosomal segments
loss of chromosomal segments (90%, 80%, 70%, 60% or less than 50% of original segment size)
differences in heterochromatin distribution and amounts (differences of 10%, 20% or greater than 20% and/or distribution differences of heterochromatin greater than 20%)
translocation events (2 or more translocation events on the same or different chromosome segment compared to parental cell line)

Producer cells can also be characterized on the basis of overall cell phenotype, such as intracellular content of mitochondria, reactive oxygen species (ROS), mitochondria membrane potential, endoplasmic reticulum, Golgi apparatus, and so on, using the materials discussed above.

### 9. Determining Production Capacity and Characteristics of Producer Cells

A cell line or mixed cell population that has been selected for high levels of protein production may be characterized in comparison with the parental or originating cell line by any one or more of several different parameters. For example, the selected cells may have: (1) a genome that is more aneuploid than the starting cells, containing part or all of the genome of two or more parental cell lines (which may or may not be the same), (2) a higher concentration of mitochondria with a desired phenotype, endoplasmic reticulum, Golgi apparatus, or other phenotypic feature compared with any one or all of the parental cell lines (for example, between 2 to 5-fold or 4 to 8 fold, or more than 2-, 4-, or 8-fold higher), (3) higher or lower concentration of reactive oxygen species (for example, 1.5-fold, 2-fold, 3-fold, or more) depending on the cell line used to make hybrids; (4) a capacity to produce a level of target protein per cell or per liter of culture fluid that is substantially higher than the parental cell line (for example, more than 2-fold, 4-fold, or eight-fold, or between 2 to 20-fold), (5) a capacity to produce a level of target protein per cell per unit time (for example, more than 10, 20, 40, or 100 picograms per cell per day, or between 5 and 20 or 10 and 100 picograms per cell per day); (6) a capacity to produce a certain amount of protein per volume of culture fluid (for example, at least 100, 200, 500, or 1,000 milligrams per liter culture fluid). When protein productivity is compared between cells before and after fusing and sorting according to this disclosure, the protein production per cell or per culture may increase by 5-fold, 10-fold, or 15-fold. These terms are inclusive of higher performing cells. Thus, an engineered optimized cell that has increased production of 20-fold compared with the starting cell population meets all these criteria.

For the purpose of making such comparisons, the producer cell line can be compared with a standardized population of the original cell line, either kept on hand, as part of the same system, or obtained from a reference source. CHO derived producer cells may be compared with an original CHO cell line, such as the line deposited under the catalog number CRL-12023 at the American Type Culture Collection (ATCC). HEK 293 derived producer cells may be compared with HEK 293 cells deposited at the ATCC under catalog number CRL-1573. The technology of this disclosure can be distributed as a protein production system that comprises both a starting cell line, and a producer cell line derived therefrom that has one, two, or three mitochondria features such as (1) a relatively high density of mitochondria; (2) a relatively high or low reactive oxygen species (ROS) per cell; and/or (3) a relatively high or low mitochondrial membrane potential; as determined, for example, using one or more of the vital dyes listed above.

### 10. Genetically Altering and Selecting Producer Cells that Synthesize and Produce the Target Protein or a Proxy

To obtain cells that express a reporter protein or a target protein, producer cells or their precursors are transfected with a transgene that encodes it.

The expression of gene cassette(s) containing a protein encoding region can be under control of following combinations of mammalian promoters: ubiquitous, endogenous viral promoters (not ubiquitous, e.g. p5 and p19), hybrid promoters, and/or inducible promoters that cause expression of single or multiple gene cassette(s) in the host cell line. The gene can be placed in forward or reverse orientation with respect to the promoter. The gene or plurality of genes can be flanked by recombination sites (FRT and its variants; and/or lox and its variants). These recombination site variants include: loxP, lox511, lox2272, FRT or mFRT71. Site specific recombinases such as Cre or Flippase is expressed in the same cell to allow for site-specific recombination and change the orientation of the gene from reverse to forward.

For example, the recombinase can be expressed by transient transfection wherein the gene encoding for the recombinase is under the control of ubiquitous or inducible mammalian promoter. In another illustration, purified recombinase protein or mRNA can be transfected into the cell. In another illustration, the recombinase can be delivered using adenovirus, lentiviruses, AAV, Moloney Murine Leukemia Virus (MMLV), Murine Stem Cell Virus (MSCV), Vesicular Stomatitis Viruses (VSV), or Herpes Simplex Viruses (HSV). Multiple genes can be expressed by the same promoter through the use of polycistronic elements such as T2A, P2A, E2A, F2A, IRES and IRES2 elements. Inducible promoters result in expression of gene or gene cassette upon addition of a stimulus, that can be chemical (e.g. doxycycline, tetracycline, cumate, recombinase such as Cre or Flippase) or physical (e.g. blue light). The level of production of the target protein can be determined in the course of processing using a transient transfection method to insert a gene expression cassette.

Alternatively or subsequently, permanent transfection can be done that integrates the gene of interest and/or a marker gene into the genome of the cell line. Multiple copies of gene integration (as many as fifty of integrated copies per cell) can be achieved by co-transfection of transposase and gene cassette flanked by transposase recognition sites known as transposase inverted terminal repeats.

Transfection can be done using liposome-based reagents (for example, Lipofectamine™ 3000, Expifectamine 293, FuGENE™ HD, X-Fect nanoparticles polymer, Trans-IT Pro reagents, Trans-IT VirusGen, polyethylenimine), calcium phosphate, electroporation, or infection with an adenovirus, retrovirus or lentivirus-based vector.

Following transfection, the cells are tested for production for packaging of the intended target protein: for example, by enzyme-linked immunosorbent assay (ELISA), quantitative real-time PCR (qPCR), or biolayer interferometry (BLI). Cells or clones having increased production of the desired target protein are selected. The objective can be an increase in production of the target protein that is 1.5, 2, 4, 8, 12, 16, 20, or 100-fold higher than the parental cell line.

In principle, transfection of a cell with a transgene can be done either before, during, or after one or more cycles of fusion and selection for other features, as put forth in this disclosure. For example, the fusion and selection can be done before transfection with a transgene encoding the target protein, thereby establishing a parental cell line suitable for high-level production. Alternatively, the transfection can be done into the originating parental cell line containing gene(s) of interest and used to track production levels during subsequent fusion and sorting steps, or to provide another basis for such sorting. Alternatively, the transfection can be done as an intermediate step, wherein the cells have already been subject to one or more cycles of fusion and selection for some other feature such as ER, Golgi, and mitochondria phenotype (referred to earlier in this disclosure). The resulting hybrid is transfected to express the target protein, and then subjected to further cycles of fusion and selection for increased production and/or other features referred to earlier in this disclosure.

Another option is to develop a cell line using a reporter gene for a marker protein as a proxy for the target protein. The marker protein could be, for example, an enzyme that coverts a soluble substrate to a visible marker Such enzymes include horse radish peroxidase, soybean peroxidase, alkaline phosphatase, beta-galactosidase, hematin, acacia, iron porphyrins, and peroxidase-mimicking nanomaterials (nanozymes). The soluble substrate for a peroxidase could be for example, 3,3′,5,5′-tetramethylbenzidine (TMB), luminol (5-amino-2,3-dihydrophthalazine-1,4-dione), ABTS (2,2′-azinobis [3-ethylbenzothiazoline-6-sulfonic acid]-diammonium salt), or a combination thereof. Alternatively, the marker may be an enzyme that produces a fluorescent or light-emitting reaction product, such as a luciferin, or it could be a non-enzymatic protein that can be measured, for example, by immunoassay. This creates a parental cell line that is optimized for expression of recombinant protein generally, not necessarily focused on a particular target protein. A producer cell line is thereby established, with the expectation that the beneficial characteristics of the cell line will be retained when used to produce a selected target protein.

Ultimately, once the producer cell line is established the marker is removed or diluted out, and replaced with a transgene encoding the target protein. Transfection can again be done randomly into the genome, using the techniques listed above, and expression of the reporter gene is curtailed. Alternatively, the gene for the reporter gene can be substituted with a gene that encodes the target protein using a targeted integration technique. Such techniques comprise, for example, CRISPR/Cas, CRISPR/Cas associated transposase (CASTs), recombinase cassette exchange (RMCE), a zinc-finger recombinase (ZFR), or a transcription activator-like effector nuclease (TALEN). That way, the gene of interest is inserted into the genome of the cells from the producer cell line or the mixture at a location that is pre-selected as permitting or supporting a high level of transcription, compared with other locations in the genome.

The technology of this disclosure can be used to produce products and pharmaceutical agents that have more than one polypeptide chains: for example, a specific antibody, comprising a heavy chain and a light chain. While the components can be produced in separate cell lines and assembled subsequently, it is often preferable to produce all components in the same producer cell, whereby the entire complex can be assembled before secretion into the medium.

In this case, the cells can be transfected sequentially or simultaneously with separate transgenes that expresses each of the polypeptide chains. Alternatively, the cells can be transfected with a single transgene that has separate encoding regions for each of the polypeptide regions, controlled so as to be expressed as separate polypeptides, or together as a fusion protein which is subsequently separated into the several polypeptide components. At some point in the process, the producer cell line is obtained by a process that comprises selecting cells that express each and all of the polypeptide components at a high capacity compared with the starting cell population.

### 11. Promoters for Expressing a Transgene that is Stably Transfected into Producer Cells

The transgene encoding the target protein can optionally be placed under control of a strong constitutive protein, or under control of an inducible promoter, depending on whether the user wishes to regulate protein expression at various stages in preparation and operation of the production culture.

An example is the cumate inducible promoter (CymR), a repressor that binds to the cumate operator sequences (CuO) in the absence of Cumate. In presence of cumate, Cumate binds to CymR allowing for activation of gene downstream of CuO. U.S. Pat. Nos. 8,728,759 and 7,745,592 B2. Also suitable are tetracycline response elements (TRE), which can be induced using doxycycline or tetracycline. Light inducible promoters can also be used, such as the blue light inducible promoter from GenTarget, Inc.

### 12. Biological Drugs that can be Produced Using this Technology

The technology put forth in this disclosure can in principle be used to produce any protein that is used as a pharmaceutical ingredient. Categories include the following:
Monoclonal antibodies that target specific proteins or cells (used, for example, in cancer therapy, autoimmune disease, and infectious disease);
Proteins used to replace or supplement physiological proteins that are deficient: such as hormones (insulin, growth hormone, and erythropoietin), and enzymes (for example, in lysosomal storage disease)
Protein components of vaccines, such as influenza and hepatitis B;
Blood components, such as Factor VIII and Factor IX

At the time of this writing, the top 10 monoclonal antibodies used as pharmaceuticals (in terms of market size) are as follows:
adalimumab (Humira®)
rituximab (Rituxan®/MabThera®)
trastuzumab (Herceptin®)
bevacizumab (Avastin®)
infliximab (Remicade®)
pembrolizumab (Keytruda®)
etanercept (Enbrel®)
nivolumab (Opdivo®)
cetuximab (Erbitux®)
omalizumab (Xolair®)

Drugs that are recombinant proteins but not antibodies are exemplified by the following:
insulin: Humulin®, Novolin®
erythropoietin: Epogen® Procrit®
human growth hormone: Genotropin®, Humatrope®
interferon-beta: Betaseron®, Avonex®, Rebif®
tissue plasminogen activator (tPA): Activase®
granulocyte colony-stimulating factor (G-CSF): Neupogen®, Zarxio®
thrombopoietin receptor agonists: Nplate®, Promacta®
follicle-stimulating hormone (FSH): Gonal-F®, Follistim®
Factor VIII: Advate®, Kogenate®
Factor IX: BeneFIX®
parathyroid hormone (teriparatide): Forteo®

### 13. Formulation of Medicaments

Preparation and formulation of pharmaceutical agents for use according to this disclosure can incorporate standard technology, as described, for example, in the most recent edition of Remington: The Science and Practice of Pharmacy, and in the most recent edition of Stefan Behme's Manufacturing of Pharmaceutical Proteins. The formulation will typically be optimized for administration systemically, either intramuscularly or subcutaneously, or for administration orally or nasally (for example, to stimulate the mucosal immune system).

Preparations of a pharmaceutical protein may be provided as one or more unit doses (either combined or separate), each containing an amount of the pharmaceutical agent that is effective in the treatment of a chosen disease, infection, or clinical condition. The commercial product may contain a device such as a syringe for administration of the agent or composition in or around the target tissue of a subject in need thereof. The product may also contain or be accompanied by an informational package insert describing the use and attendant benefits of the target protein in treating the condition for which it is indicated and approved.

### 14. Other Commercial Uses of Recombinant Protein Manufactured According to this Disclosure

The producer cells and other technology of this disclosure can be used to manufacture recombinant proteins for any industrial process as a substitute for or improvement on proteins from other sources. Such industrial processes include but are not limited to the food industry, biofuels, plastics, lubricants, surfactants, solubilizers, dispersion enhancers, coatings, ceramics, ink, textiles, and agricultural feed.

In the manufacture of biofuels from biomass, there are the following industrial enzymes: cellulase, hemicellulase, amylase, pectinase, and GH61 protein (U.S. Pat. No. 8,298,759). In the manufacture of detergents, there are the following industrial enzymes: protease amylase, lipase, and cellulase.

In food production, there are the following:
recombinant chymosin in cheese production (fermentation-produced chymosin, FPC) as an alternative to traditional animal-derived rennet in cheese production
recombinant enzymes in baking, such as alpha-amylase, to improve dough characteristics
recombinant proteins that affect taste in plant-based meat alternatives: myoglobin (Motif FoodWorks), and leghemoglobin (Impossible Foods).

### EXAMPLES

### Example 1: Production of Protein by Cell Hybrids

In this example, CHO cells were fused and sorted for a high content of endoplasmic reticulum (ER) for the purpose of maximizing protein production.

CHO-K1 cells were exposed to a PEG-assisted fusion procedure. The cells were allowed to recover for one week, then the procedure was repeated for a total of three times. Following recovery from the third fusion, the cells were stained with vital ER-tracking dye (ER-Tracker™ Green (glibenclamide BODIPY® FL); Invitrogen, E34251) and sorted using a FACSAriaII™ cell sorter (BD Biosciences). Ten percent of the viable population exhibiting the highest amount of staining with ER-Tracker dye was collected. Following a two-week recovery in culture, the cells were exposed to a final fusion, stained with ER-tracking dye, and analyzed using a LSRII™ flow cytometer (BD Biosciences).

To measure protein production in the fused cells, and the parental CHO population, the cells were transfected to express secreted alkaline phosphatase (SEAP). The transfection was performed as follows:
1. Centrifuge 10⁶ cells.
2. Discard supernatant
3. Resuspend in 100 μL Cell Line Nucleofector™ Solution T
4. Add 2 μg SEAP expression plasmid
5. Transfer to electroporation cuvette
6. Electroporate using Amaxa™ Nucleofector II and preset program U-023
7. Add 0.5 ml growth medium
8. Transfer cells into 6-well plate containing 1 mL. growth medium per well

FIG. 4 shows the results (specific productivity of secreted alkaline phosphatase). The expression of the marker protein (SEAP) in the fused cells shows over 4-fold improvement.

### Example 2. Demonstration of High Level Production of Viruses from Fused Cells

This example shows results from experiments in which fused cells were used for producing viral particles rather than recombinant protein.

Fusion of HEK 293 cells was performed multiple times using PEG as a fusogenic agent to form autotypic hybrids (a plurality of cells from one cell line). For packaging and production of adeno-associated viruses, serotype 2 (AAV2), un-engineered and engineered HEK 293F were transfected with a helper plasmid, a virus plasmin containing AAV Rep and Cap proteins, and a transfer plasmid expressing NeonGreen fluorescent protein under the control of a constitutive cytomegalovirus (CMV) early promoter, flanked by AAV2 inverted terminal repeats (ITRs), and an additional plasmid expressing microRNA, mi342 under the control of a ubiquitous CMV promoter. Transfections were performed using linear polyethyleneimine (PEI). Post-transfection, crude virus was extracted from cell lysates and virus was recovered by centrifugation. Quantitative PCR was used to measure viral copy number produced by un-engineered and engineered HEK 293 cell lines. Details of the protocol are put forth in international patent application PCT/US2023/024973.

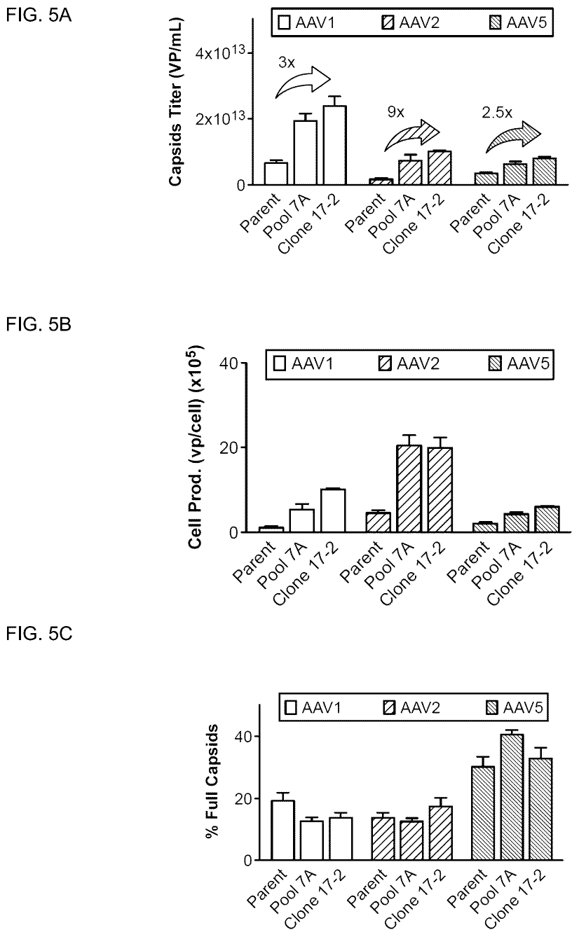

FIGS. 5A, 5B, and 5C demonstrate higher productivity of viral particles from cells fused, cloned, and sampled. Host cells were transiently transfected with (1) transfer plasmid expressing fluorescent reporter; (2) packaging plasmid expressing Rep and Cap proteins specific for AAV1, AAV2, or AAV5; and (3) helper plasmid. Capsid concentration or titer were measured by biolayer interferometry (BLI) using a biosensor that binds to AAV1, AAV2, or AAV5 capsids. Cumulative capsids productivity (FIG. 5A), cell specific productivity (VP/cell) (FIG. 5B), and percent full capsids (FIG. 5C) of AAV1, AAV2, AAV5 of HEK 293 parent, engineered pool (7A) and clone (#17-2). Engineered clone (17-2) showed 3-fold, 9-fold, and 2.5-fold increase compared with parent host, respectively.

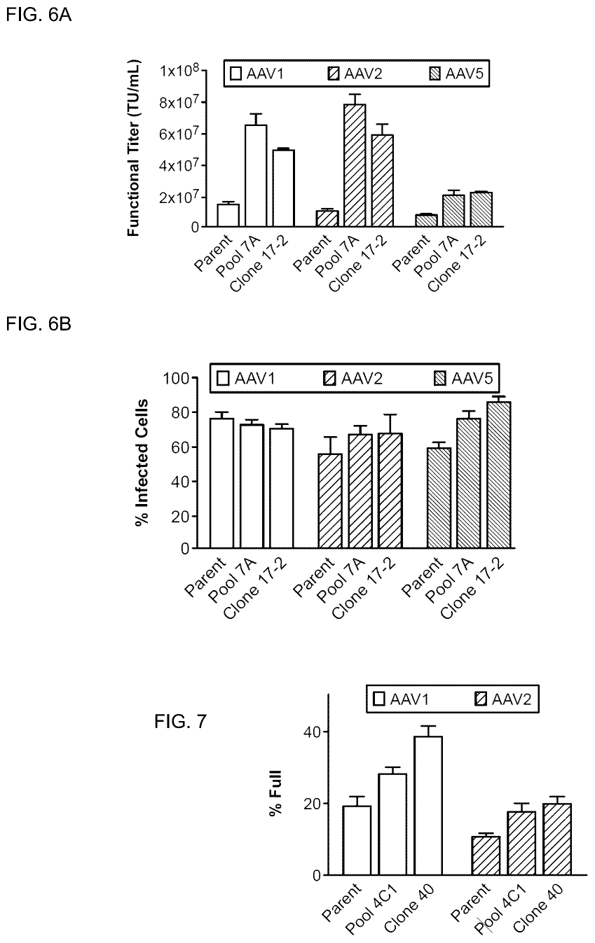

FIGS. 6A and 6B demonstrate higher functional titer from cells fused, cloned, and sampled according to this illustration. Concentrated packaged AAV1, AAV2, and AAV5 virus were infected in HEK 293T cells at different multiplicity of infection (MOI) and percent infected cells were determined by gating for fluorescent-positive cells using flow cytometry. FIG. 6A: Mean functional titer were calculated at three different virus concentrations (dilutions ranging from 1:25 to 1:300). FIG. 6B: Cell infectivity measured by % cells exhibiting GFP fluorescence (AAV transfer vector transgene) at 3 different multiplicity of infection (MOI) ranging from 500 to 50,000 (serotype dependent).

FIG. 7 demonstrates an improvement in the proportion of capsids containing a nucleic acid payload achieved in this illustration. Host cells were transiently transfected with transfer plasmid expressing fluorescent reporter; packaging plasmid expressing Rep and Cap proteins specific for AAV1 or AAV2; and helper plasmid. Full-to-empty ratio of AAV1 and AAV2 of lysed cells were measured by BLI. Engineered HEK 293T pool (4C1) clone (#40) selected for high mitochondria (top 10%) and high reactive oxygen species (top 10%) showed a two-fold increase in full-to-empty ratio.

### Example 3: Suitable Protocol for Selection of Fused Cells with High Mitochondria and High Reactive Oxygen Species

In the course of obtaining the AAV producing cells in Example 2, cell hybrids were screened for different phenotypes of mitochondria and reactive oxygen species (ROS), hybrids were stained with CellROX® Deep Red Reagent, a fluorogenic probe for measuring cellular oxidative stress in cells; TMRM (tetramethyl rhodamine methyl ester), which measures the membrane potential of mitochondria in living cells, and Biotracker 405 Blue Mitochondria, which stains the mitochondria membrane. LIVE/DEAD Fixable NIR (Thermo Fisher) was used in this experiment to stain for live cells.

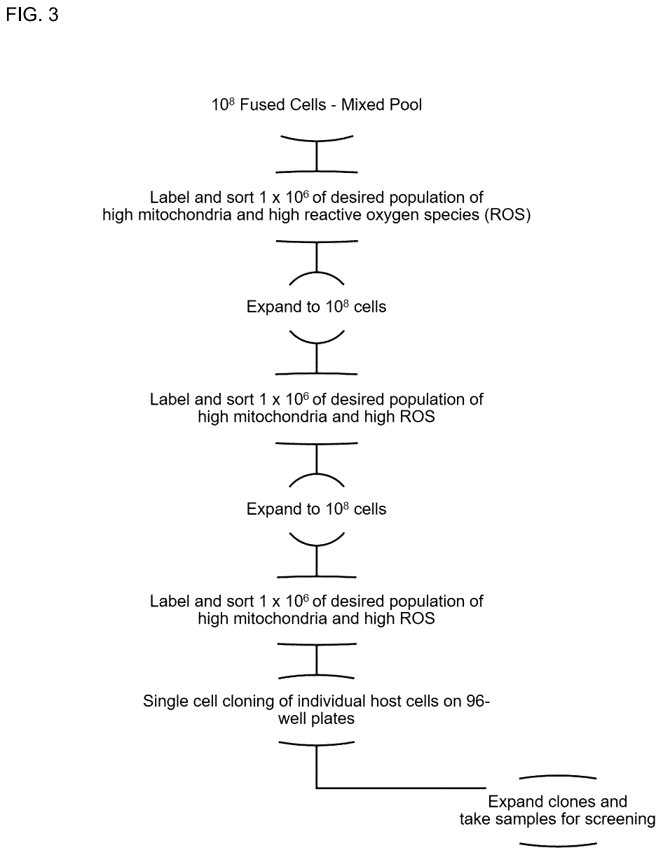

FIG. 3 shows the workflow used. Fused cells were cultured in complete, animal origin free (AOF), chemically defined cell culture medium: CDM4 PerMAb plus L-glutamine and detached using StemPro™ Accutase™ Cell Dissociation Reagent. Samples of cells were combined with a calculated volume of each dye to final concentrations in 200 mL of cell suspension containing 1×10⁸ cells in a 500 mL shake flask. Samples were incubated in shaker overnight at 37° C. with 8% CO₂.

For cell sorting, 200 mL of cell sample was centrifuged at 300×g for 5 min. The cell pellet was suspended in Accutase cell dissociation reagent, diluted, and strained into a sterile 50 mL centrifuge tubes. Cells were sorted using a Sony SH800S Cell Sorter with the following gates: Gate 1—cell ID gate; Gate 2—singlets gate; Gate 3—live cells gate; Gate 4—Biotracker 405 Blue Mitochondria (select the top 10%); Gate 5—TMRE×CellROX Deep Red (select the top 10% quadrant).

Populations of 500,000 sorted cells were expanded for 4 to 5 days and used for single-cell cloning in 96-well plates containing 150 μl of medium per well. Once individual wells were 80% confluent, they were expanded stepwise to 125 mL shake flasks, and used to create cell banks.

### Example 4: Detailed Procedure that was Used to Obtain Protein Producer Cell Lines by Selecting Cells that have a Preferred Phenotype of Mitochondria

The data in FIG. 2A were obtained using the CHO-K1 cell line obtained indirectly from the American Type Tissue Collection (ATCC). The cells were fused to create hybrids using polyethylene glycol (PEG) 1500 as fusogenic agent. 10⁸ cells were exchanged into EX-CELL CD CHO Fusion® medium (Millipore Sigma) containing L-glutamine, and gently centrifuged. The cells were suspended in medium containing 50% PEG 1500. incubated briefly, then fresh medium was added in stages. After a further incubation, the cells were centrifuged, washed, and resuspended in fresh medium, thereby obtaining a population that contained cell hybrids and a proportion of unfused cells.

The cells were then screened for a high intracellular content of endoplasmic reticulum (ER). Cells were stained with ER Tracker Green dye overnight. The top 10% of stained cells were obtained by cell sorting, allowed to recover, and expanded in shake flasks until the total number of cells was again greater than 10⁸. The selected cells were then fused again using PEG 1500. Five cycles of cell fusion, ER sorting, and expansion were done consecutively, selecting the highest 10% of stained cells each time. After the fifth fusion, the cells were cloned. Single cells of the top 10% ER expressors were deposited into 96-well plates in Ex-Cell CD Fusion medium containing L-glutamine plus 5% InstiGRO™ CHO supplement (Advanced Instruments), and expanded.

Next, a sample was taken from each of these clones and tested for protein expression by transient transfection, using trastuzumab as the marker protein. Two expression vectors were used at a molar ratio of 2 to 1. Vector 1: CMV promoter, trastuzumab heavy chain encoding region, SV40 poly A tail. Vector 2: CMV promoter, trastuzumab light chain encoding region, SV40 poly A tail. The transfection was done as follows:
1. Centrifuge 10⁸ cells.
2. Discard supernatant
3. Resuspend in 25 mL of transfection medium.
4. Prepare diluted DNA: 48 μg of DNA in 2.5 mL of Opti-MEM I
5. Add 100 μL of Trans-IT Pro polymer.
6. Wait 5-15 minutes at room temperature for complex to form.
7. Add complex to cells.
8. Incubate cells at 37° C., 8% CO₂ for up to 14 days.
9. Harvest supernatant at Day 14.

The transfected cells from each sample were expanded in culture for two weeks, and trastuzumab concentration was measured in the medium. Antibody concentrations were determined using biolayer interferometry with Protein A sensors (Gator Bio) that bind to the Fc portion of the antibody. Cell culture supernatant was diluted 1:4 before measurement. A quantitative scale was established using purified trastuzumab IgG1 at known concentrations.

Having identified the highest producer clone (Clone #4) by sampling and testing, attention reverted to the non-transfected cells. Clone #4 was expanded so that the cells could be screened for mitochondria features.

The expanded Clone #4 cells were stained for mitochondria content using Biotracker 405 Blue Mitochondria, selected for low reactive oxygen species (ROS) using CellROX Green, and selected for low mitochondria membrane potential using tetramethylrhodamine ethyl ester perchlorate (TMRE). Cells were sorted using a Sony model SH800S Cell Sorter with the following gates: Gate 1—cell ID gate; Gate 2—singlets gate; Gate 3—live cells gate; Gate 4—Biotracker 405 Blue Mitochondria (select the top 10%), Gate 5—TMRE×CellROX Green (select the bottom 10% quadrant, low expressors for TMRE and Cell ROX).

Cells with the chosen mitochondria phenotype were expanded again, recloned, and re-screened for protein expression. Each clone was sampled, and the samples were transfected to express trastuzumab, as before. The highest protein producer clone from the second cloning was banked in untransfected form.

The data in FIG. 2B were obtained using the HEK 293T cell line obtained from the ATCC. The cells were fused using polyethylene glycol (PEG) 1500 as before. Seven cycles of cell fusion and expansion were done consecutively, this time without screening for high levels of ER.

After the seventh fusion, the cells were expanded, and screened for a mitochondrial phenotype: specifically, high ROS and high mitochondria membrane potential. Biotracker 405 Blue mitochondria, TMRE, and CellROX Deep Red was added to the cells and incubated overnight in cell culture medium. Cells were prepared for sorting by centrifuging, suspended in Accutase cell dissociation reagent, diluted, and strained. Cells were sorted using a Sony SH800S Cell Sorter with the following gates: Gate 1—cell ID gate; Gate 2—singlets gate; Gate 3—live cells gate; Gate 4—Biotracker 405 Blue Mitochondria (select the top 10%), Gate 5—TMRE×CellROX Deep Red (select the top 3% quadrant).

Positively selected cells were expanded and cloned n 96-well plates. Once wells were 80% confluent, they were expanded stepwise, and sampled for expression screening using the two trastuzumab vectors. High producer clones including Clone #1) were banked in the untransfected state. The results were as follows:

FIG. 2A was obtained from CHO cells that went through five rounds of fusion and selecting for high ER, followed by selection for high mitochondria level and low reactive oxygen species (ROS). The graph shows cumulative productivity (mg/L) of trastuzumab following transient transfection into (1) the starting population of CHO cells; or (2) the final clone of fused and selected CHO cells (n=3). Selecting for high ER, high mitochondria and low reactive oxygen species yielded a 15-fold improvement compared with the un-engineered parent cell line.

FIG. 2B was obtained from HEK 293 cells that went though seven rounds of fusion, followed by selection for high mitochondria level and high instead of low ROS. The graph shows cumulative productivity (mg/L) of trastuzumab from (1) the starting population of HEK 293 cells; or (2) the final clone of fused and selected HEK 293 cells (n=3). In this instance, selecting for high mitochondria and high ROS yielded a 10-fold improvement in protein produced. There was no screening for high ER. This shows that screening for an optimal mitochondria phenotype alone is sufficient to obtain hybrid producer cells with increased protein production capacity.

### INCORPORATION BY REFERENCE

For all purposes in the United States of America, each and every publication and patent document referred to in this disclosure is incorporated herein by reference in its entirety for all purposes to the same extent as if each such publication or document was specifically and individually indicated to be incorporated herein by reference.

### PRACTICE OF THE CLAIMED INVENTION

The technology provided in this disclosure and its use are described within a hypothetical understanding of general principles of cell culture and pharmaceutical manufacture. These discussions are provided for the edification and interest of the reader, and are not intended to limit the practice of the claimed invention. All of the products and methods claimed in this application may be used for any suitable purpose without restriction, unless otherwise indicated or required.

While this disclosure has been described with reference to the specific embodiments, changes can be made and equivalents can be substituted to adapt this disclosure to a particular context or intended use as a matter of routine experimentation, thereby achieving benefits of this disclosure without departing from the scope of what is claimed.

The invention claimed is:

1. A method of producing a target protein, the method comprising:
providing a starter population of cultured cells;
forming cell hybrids from the starter population, each comprising two or more cells;
enriching the cells for a particular mitochondria phenotype;
genetically altering the cells to express the target protein; and
culturing the genetically altered cells to produce the target protein.

2. The method of claim 1, wherein the selected mitochondria phenotype comprises a high or low content of mitochondria per cell, a high or low reactive oxygen species (ROS) per cell, and/or a high or low mitochondria membrane potential per cell.

3. The method of claim 1, further comprising enriching the cells for a high content of endoplasmic reticulum per cell.

4. A process for establishing a producer cell line for high efficiency production of a target protein, the process comprising:
(a) providing a starter population of cultured cells;
(b) forming a population of cell hybrids from the starter population, each comprising two or more cells;
(c) testing the cell hybrids for mitochondria phenotype, including mitochondria content per cell, reactive oxygen species (ROS) per cell, and/or mitochondria membrane potential per cell;
(d) selecting cell hybrids that have a higher or lower content of mitochondria per cell, reactive oxygen species (ROS) per cell, and/or mitochondria membrane potential per cell compared with other cells in the population of cell hybrids and
(e) culturing and expanding cell hybrids selected in step (d), thereby establishing said producer cell line.

5. The process of 4, wherein step (d) comprises selecting cell hybrids that have a higher content of mitochondria per cell.

6. The process of claim 4, wherein step (d) comprises selecting cell hybrids that have a higher or lower content of reactive oxygen species (ROS) per cell.

7. The process of claim 4, wherein step (d) comprises selecting cell hybrids that have a higher or lower mitochondria membrane potential per cell.

8. The process of claim 4, further comprising selecting cell hybrids that have a higher density of endoplasmic reticulum or Golgi apparatus per cell.

9. The process of claim 4, further comprising cloning the cells and selecting clones with high protein production capacity per cell.

10. The process of claim 4, further comprising genetically altering the cells or a sample thereof to express a transgene that encodes a recombinant protein before and/or after step (c), and selecting cell hybrids or clones thereof that produce more of the recombinant protein per cell compared with other cell hybrids or clones.

11. The process of claim 10, wherein the recombinant protein is a maker protein, or a particular target protein intended for commercial manufacture.

12. The process of claim 4, further comprising selecting cell hybrids that have a faster growth rate than other cell hybrids.

13. The process of claim 4, wherein the starter population is a plurality of cells from a single cell line.

14. A producer cell line adapted for high efficiency production of protein, established according to the process of claim 4.

15. The producer cell line of claim 14, which has been genetically altered to express a transgene encoding a particular target protein.

16. A line of producer cells wherein each cell is a hybrid of one or more parental cell lines, wherein the producer cells comprise an expressible transgene that encodes a target protein, and contain more mitochondria and/or reactive oxygen species (ROS) per cell than any of the parental cell lines.

17. The process of claim 4, wherein the target protein is a pharmaceutical agent or portion thereof selected from an antibody molecule, a therapeutic enzyme, a hormone, a growth factor, or a protein that is a naturally occurring component of blood.

18. The process of claim 4, wherein the target protein is a food ingredient or an industrial enzyme.

19. A method of manufacture, comprising culturing cells from the producer cell line of claim 15 to produce said target protein, wherein the target protein is a pharmaceutical agent.

20. The method of claim 19, further comprising purifying the target protein from the cell culture, and compounding the target protein to produce the pharmaceutical product in a manner such that the product is suitable for human administration.
